(19) United States
(12) Patent Application Publication
(10) Pub. No.: US 2014/0106461 A1
(43) Pub. Date: Apr. 17, 2014

(54) MERCURY MONITORING SYSTEMS AND METHODS

(71) Applicants: Steve T. Gunther, Omaha, NE (US); Joel Creswell, Seattle, WA (US); Colin Davies, Seattle, WA (US)

(72) Inventors: Steve T. Gunther, Omaha, NE (US); Joel Creswell, Seattle, WA (US); Colin Davies, Seattle, WA (US)

(73) Assignee: Brooks Rand Inc, Seattle, WA (US)

(21) Appl. No.: 14/042,391

(22) Filed: Sep. 30, 2013

Related U.S. Application Data
(60) Provisional application No. 61/707,770, filed on Sep. 28, 2012.

(51) Int. Cl. G01N 33/20

(52) U.S. Cl. CPC G01N 33/203
USPC 436/81; 422/78

(57) ABSTRACT

Mercury monitoring system and methods for detecting an amount of total mercury in a liquid sample include a sample inlet for receiving a liquid sample, a heating chamber in direct fluid communication with the sample inlet, an oxidation chamber for oxidizing the evaporated sample, a mercury amalgamator for trapping elemental mercury, and may include a mercury detector.

### CROSS-REFERENCE TO RELATED APPLICATION

This application claims the benefit of U.S. Provisional Application No. 61/707770, filed Sep. 28, 2012, the disclosure of which is hereby expressly incorporated by reference in its entirety.

### STATEMENT OF U.S. GOVERNMENT INTEREST

This patent application was made with U.S. Government support under the Small Business Innovation Research (SBIR) Award granted by the Department of Energy (DOE). The U.S. Government may have certain rights in the patent application or in the claimed inventions.

### BACKGROUND

Mercury is a hazardous pollutant that threatens human and ecosystem health and exists in surface water and groundwater environments. Monitoring mercury in water and many other environmental matrices is challenging due to the considerable effort and expense involved in collecting samples, maintaining sample integrity during transport and storage, and subsequent laboratory analysis. These constraints often make high frequency sampling infeasible and limit opportunities for long-term monitoring. Because samples must be analyzed in the laboratory, collection of real-time data is difficult. Yet mercury loading to surface water systems and mercury export from subsurface systems, including those in contaminated areas, is often episodic, with the majority of mercury contributions coming during storm events. In such dynamic environments, high frequency and/or real-time monitoring would be helpful to accurately differentiate between groundwater and surface watershed inputs, and to gain an accurate understanding of mercury levels.

While automated samplers may allow for unattended, high frequency sampling, these systems are limited in their usefulness because of the need to store samples in open containers until they can be manually sealed by field personnel, resulting in a high risk of atmospheric contamination and sample cross-contamination. These systems also do not reduce the expense, time, or possible sample integrity changes associated with transporting samples back to the laboratory for analysis. Because samples must be collected from these systems by field personnel, it is difficult to deploy them in remote sites, far from analytical facilities.

Because groundwater transport of contaminant plumes and subsurface contaminant geochemistry occur on long time scales, sometimes on the order of decades or more, long-term monitoring may be required in order to adequately characterize or decontaminate a system. The expense and effort involved in field-based mercury monitoring mentioned above make it difficult to carry out long-term research on subsurface mercury contamination.

Therefore, there exists a need for systems and methods for monitoring mercury that are capable of high-frequency sampling, eliminate the need for transportation of samples back to a laboratory for analysis, and can operate unattended and at low cost for long periods of time. While some portable mercury analyzers may be currently available, they lack sufficient sensitivity to detect environmentally relevant mercury concentrations in most systems. Because of their low sensitivity, these systems have not been adapted for long-term deployment or unattended monitoring of environmental systems. In addition to field deployable systems, there exists a need for improved systems and methods for monitoring mercury in lab analyses.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a mercury monitoring system for detecting an amount of total mercury in a liquid sample is provided. The system generally includes a sample inlet for receiving a liquid sample, and a heating chamber in direct fluid communication with the sample inlet. The heating chamber is configured for evaporating the entire liquid sample for detection in a single heating cycle, the heating chamber including a fluid reservoir to receive and contain the entire liquid sample prior to evaporation. The system further includes an oxidation chamber for oxidizing the evaporated sample, a mercury amalgamator for trapping elemental mercury, and a mercury detector.

In accordance with another embodiment of the present disclosure, a mercury monitoring system for detecting an amount of total mercury in a liquid sample is provided. The system generally includes a sample inlet for receiving a liquid sample, and a heating chamber in direct fluid communication with the sample inlet. The heating chamber is configured for evaporating the entire liquid sample for detection in a single heating cycle, the heating chamber including a includes a plumbing trap for receiving the liquid sample, wherein the plumbing trap includes an inlet line at a first elevation, a fluid reservoir at a second elevation lower than the first elevation, and an outlet line at a third elevation higher than the second elevation. The system further includes an oxidation chamber for oxidizing the evaporated sample, a mercury amalgamator for trapping elemental mercury, and a mercury detector.

In accordance with another embodiment of the present disclosure, a method of detecting an amount of total mercury in a liquid sample is provided. The method generally includes collecting a liquid sample in an inlet line, transferring the entire liquid sample from the inlet line directly to the sample decomposition chamber, and containing the liquid sample in the sample decomposition chamber, to allow a flow of gas to flow from the gas source into the heating chamber when the liquid sample is contained. The method further includes heating the liquid sample to evaporate the liquid, transferring the evaporated sample through a catalytic oxidation chamber to remove combustion products, and trapping the volatilized mercury.

In accordance with any of the systems or methods described herein, further including a flow of gas delivered to the heating chamber from a gas source.

In accordance with any of the systems or methods described herein, the sample inlet may be a sample injection system.

In accordance with any of the systems or methods described herein, the sample inlet may be configured to receive a fixed volume sample.

In accordance with any of the systems or methods described herein, the fixed volume sample may have a volume selected from the group consisting of in the range of about 1.5 mL to about 10 mL and in the range of about 1.5 mL to about 20 mL.

In accordance with any of the systems or methods described herein, the fluid reservoir may include a plumbing trap for receiving the liquid sample.

In accordance with any of the systems or methods described herein, the plumbing trap may include an inlet line at a first elevation, a fluid reservoir at a second elevation lower than the first elevation, and an outlet line at a third elevation higher than the second elevation.

In accordance with any of the systems or methods described herein, the heating chamber may be configured to contain the entire liquid sample prior to evaporating the liquid sample.

In accordance with any of the systems or methods described herein, the heating chamber may not receive a sample contained in a boat.

In accordance with any of the systems or methods described herein, the heating cycle may include a first sample receipt temperature and a second sample evaporation temperature.

In accordance with any of the systems or methods described herein, the heating cycle further including a third sample decomposition temperature.

In accordance with any of the systems or methods described herein, the system may not use reagents for sample decomposition.

In accordance with any of the systems or methods described herein, further including an internal calibration system.

In accordance with any of the systems or methods described herein, the mercury detector may be a CVAFS detector.

In accordance with any of the systems or methods described herein, the mercury amalgamator may be a gold amalgamation trap.

In accordance with any of the systems or methods described herein, further including a dryer for water vapor removal.

In accordance with any of the systems or methods described herein, further including decomposing any non-volatile components in the sample.

In accordance with any of the systems or methods described herein, further including decomposing any non-volatile components in the sample.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic of a system for measuring total mercury in liquid in accordance with one embodiment of the present disclosure;

FIG. 2 is a schematic of a system for measuring total mercury in liquid in accordance with another embodiment of the present disclosure;

FIG. 3 is a more detailed schematic of the system of FIG. 2; and

FIG. 4 is a schematic for a software control program for the system of FIG. 1 or 2.

### DETAILED DESCRIPTION

The detailed description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result.

In the following description, numerous specific details are set forth in order to provide a thorough understanding of exemplary embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein.

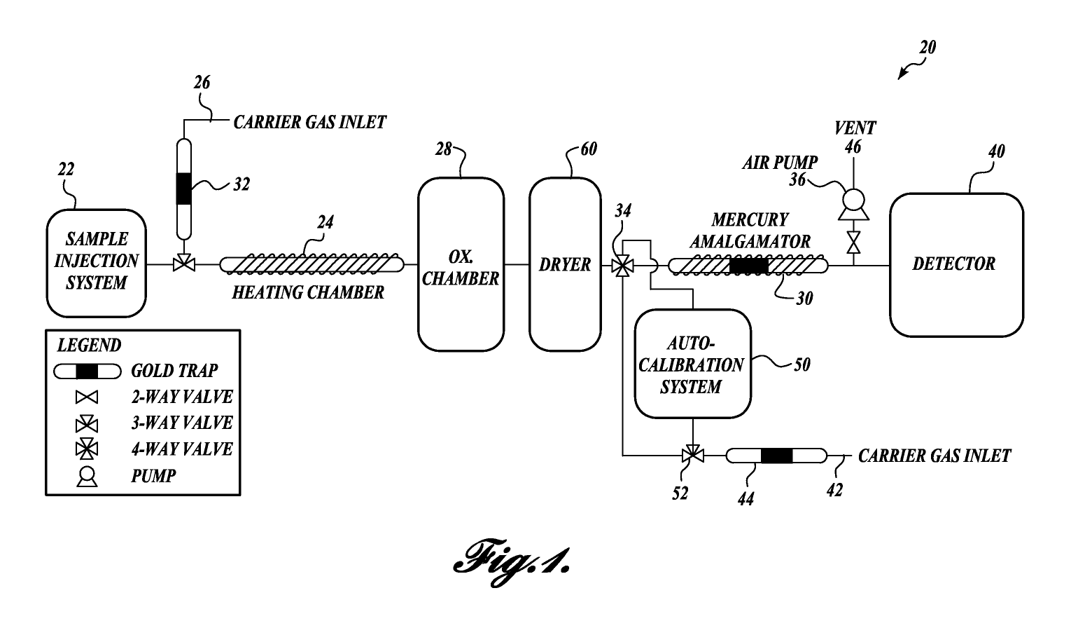

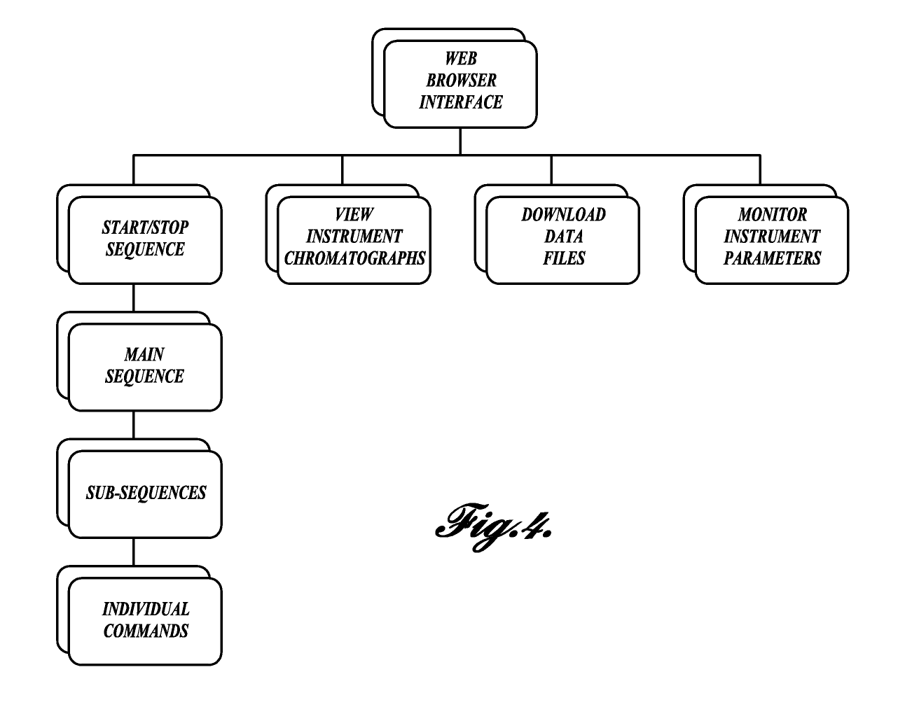

Systems and methods for monitoring mercury are provided in accordance with embodiments of the present disclosure. Referring to FIG. 1, in one embodiment of the present disclosure, a system 20 for measuring total mercury in a liquid sample, such as water or an aqueous medium, by sample collection, thermal decomposition, oxidation of combustion products, pre-concentration of mercury species by a mercury amalgamation trap, and detection. This system 20 allows for unattended, reagent-free analysis and does not require any sample pre-digestion. In addition, the system 20 may be capable of storing data locally or transmitting it, via cellular or satellite, wired, or wireless data networks, to a remote location (see, e.g., FIG. 4 for a schematic of a software control program for the system).

The system 20 makes possible several new applications in the field of mercury analysis: (1) field measurements of mercury concentrations in aquatic systems; (2) unattended monitoring of mercury concentrations in aquatic systems; and (3) measurements in an aquatic system in an industrial setting, such as an industrial plant. In addition, the system described herein may help simplify and improve the reliability of attended lab-based monitoring. The mercury monitor systems and methods described herein make possible high frequency, long term, and low cost measurements of mercury in groundwater and surface water, addressing all of the research needs described above.

In accordance with embodiments of the present disclosure, the system 20 may be a field-deployed system, a portable system, an specific site-deployed system, and/or a lab-based system. In the case of a field- or site-deployed system, the system 20 may be designed to run continuously or semi-continuously to take periodic samples from the liquid system being monitored. The system 20 may be capable of sampling and monitoring mercury from a single source or from multiple sources (for example, influent and effluent at a water treatment plant, alternating between the two or more water sources).

In the case of a lab system, the samples may be processed, for example, through an auto sampler by the operator, and the liquid may then be automatically transferred from the auto sampler containers to the decomposition chamber. This approach is advantageous in lab systems because it allows for reduced opportunities for sample contamination. This approach also allows for reduced work for the laboratory analyst, because no sample digestion or reagent addition steps are required.

A lab system that employs system components and/or method steps described herein may also allow for larger sample volumes to be thermally reduced than other thermal decomposition lab systems that are currently available. In that regard, embodiments of the present disclosure may include a water vapor removal component and/or step to reduce water vapor produced during thermal decomposition of the aqueous sample.

The system and methods described herein may further be configured to minimize power demand. As a non-limiting example a suitable power requirement may be 1000 W. It should be appreciated that the power may be sourced from one or more sources, including, but not limited to, one or more batteries, a generator, or AC power

As can be seen in the schematic of FIG. 1, the system 20 of the illustrated embodiment collects a liquid sample through a sample injection system 22, introducing it into a heating chamber 24. After the heating chamber 24 has been heated to evaporate the liquid sample, mercury-free air can be pulled from a gas source 26 by pump 36 through the heating chamber 24, carrying the gaseous sample and all dissolved gaseous mercury through an oxidation chamber 28 and then to a mercury amalgamation trap 30. After being collected, the mercury on the amalgamation trap 30 can then be thermally desorbed into the detector 40. The system 20 may also include an auto-calibration system 50, as described in greater detail below.

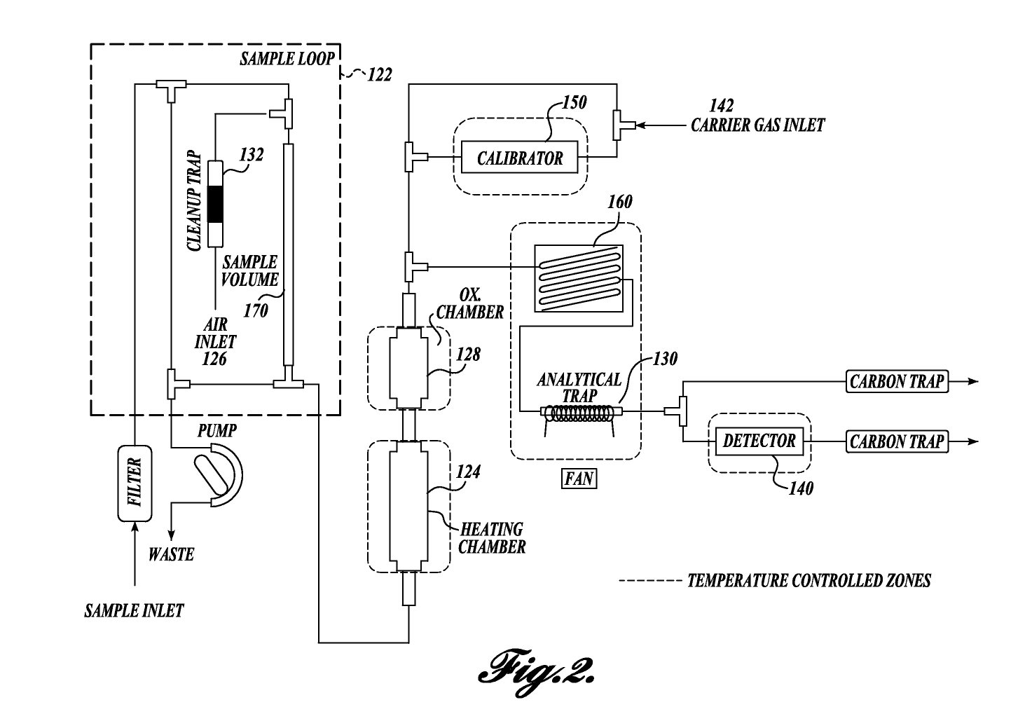

The sample injection system 22 may be configured to inject a fixed volume of sample into the system 20. In one embodiment, the sample injection system 22 is an automated system for periodic sample collection and injection. As described in greater detail below with reference to FIG. 2, the sample injection system 22 may be a sample loop system for periodic sampling.

In one embodiment of the present disclosure, the system 20 may receive liquid samples in the range of about greater than 1.5 mL to about 10 mL. In another embodiment of the present disclosure, the system 20 may receive liquid samples in the range of about greater than 1.5 mL to about 20 mL. Such high volume samples enable more precise mercury measurements and lower limits of detection.

In other previously designed analytical devices, the highest volume of liquid receivable in a single evaporation step is about 1.5 mL because of sampling technology size constraints, as well as the negative impacts of water vapor in a system from larger sample sizes. In that regard, water vapor in the system tends to inhibit mercury amalgamation and fluorescence results, as described in greater detail below. However, with such low liquid sample volumes in previously designed systems, less precise mercury measurements are obtained. In the illustrated embodiment, a pump 36 pulls the carrier gases from sources 26 and 42 to vent 46. However, it should be appreciated that the pump 36 may also be suitably located in the system 20 to push the sample from the sample injection system 22 into the other system components for processing and analysis.

The carrier gas from gas source 26 may be air, an inert gas, such as nitrogen, or a noble gas, such as argon. The use of noble or inert gases as carrier gases allows for lower detection of mercury by a CVAFS detector than by using air as a carrier gas. However, it should be appreciated that such low detection may not be required for use in the systems and methods described herein. Therefore, using mercury-free air as a carrier gas may provide for adequate detection results. Moreover, the use of air may assist in the heating and oxidation steps of the sample, while the use of a noble or inert gas may be employed during the desorption and detection steps for more precise detection results. Likewise, the gas source 42 for use in the calibration system, described below, may also be air, an inert gas, such as nitrogen, or a noble gas, such as argon.

In one embodiment of the present disclosure, an air carrier from carrier gas inlet 26 can be used for the evaporation and thermal decomposition of the sample, then an inert analytical carrier gas (such as argon) from carrier gas inlet 42 can be used to deliver the mercury from the mercury amalgamator 30 to the detector 40. This strategic use or air and an inert gas accomplishes three goals, as follows. First, the oxygen in the air aids in combustion and catalysis of the sample. Second, using air instead of argon reduces the system's operating costs and minimizes the frequency of gas cylinder changes. Third, using argon as an analytical carrier allows for high sensitivity measurements of mercury, as argon exhibits extremely low quenching of fluorescing mercury atoms. The air and argon streams from respective inlets 26 and 42 will each pass through a non-analytical gold amalgamation trap 32 and 44 prior to entering the system, as shown in FIG. 1, to ensure that both are substantially mercury-free.

After the sample has been received in the sample injection system 22, it passes to the heating chamber 24. In the heating chamber 24, the liquid sample is heated according to a heating sequence that includes sample receipt, sample evaporation, and sample thermal decomposition steps. The timing of each of the sequence steps may be based on temperature, time, or other sensors within the heating chamber 24.

In accordance with one method, the heating sequence includes heating the heating chamber 24 to a temperature below 100 degrees Celsius prior to sample injection. As a non-limiting example, a suitable temperature may be about 70 degrees Celsius. As another non-limiting example, a suitable temperature may be in the range of about 70 degrees Celsius to less than 100 degrees Celsius. This temperature range when the sample is received eliminates splashing or spurting in the system. After the sample has been received, the temperature in the heating chamber 24 can be raised to above 100 degrees Celsius to evaporate the sample. As a non-limiting example, the temperature range for sample evaporation is in the range of about 100 to about 110 degrees Celsius. As another non-limiting example, the temperature range for sample evaporation is in the range of about 100 to less than about 150 degrees Celsius. Any dissolved gaseous mercury in its volatile elemental form (Hg(0)) will evaporate and exit the heating chamber 24.

After the sample has been evaporated, the temperature in the heating chamber 24 can be raised to about 750 to 850 degrees C., preferably at least about 800 degrees Celsius, to thermally decompose any remaining non-volatile mercury species in the heating chamber 24 (Hg(II)). This high temperature heating will thermally reduce all of the non-elemental mercury species to the volatile elemental form (Hg(0)). In that regard, it is a requirement of the atomic fluorescence spectrometer that all mercury be in its elemental state (Hg(0)) for detection.

In previously designed lab analytical system, reagents are typically added to a liquid sample to cause vaporization of the non-volatile mercury compounds. However, in accordance with embodiments of the present disclosure, a heating chamber 24 is used to evaporate the entire sample (including volatile and non-volatile components) without the use of reagents. Therefore, the sample combustion technique of the present disclosure eliminates the need to add reagents to the samples. The removal of reagents from the system is important in portable or deployed systems because of the associated reagent costs, the need for reagent replenishment, and waste removal. Therefore, the elimination of reagents increases the long term deploy-ability of a field mercury monitoring system. The elimination of reagents also provides the same advantages in lab-based systems.

The system 20 described herein includes direct delivery of a liquid sample from the sample loop injection system 22 to the heating chamber 24. To enable such direct delivery, the heating chamber 24 may be specially configured such that the liquid sample does not travel through the heating chamber 24 before it can be evaporated into gaseous form.

In one embodiment of the present disclosure, the heating chamber 24 is configured with a “plumbing trap” type heating chamber, such that the sample enters the heating chamber 24 at an inlet at a first higher elevation, travels into the heating chamber at a second lower elevation and is heated. Vapor exits at an outlet, which is at a third elevation that is a higher elevation than the first elevation of the heating chamber 24. Without such a plumbing trap, the liquid sample would simply spill out of the heating chamber 24.

In accordance with one embodiment of the present disclosure, the heating chamber 24 may be configured to include a reservoir to receive the entire sample volume, leaving a head space above the liquid sample in the heating chamber 24 and a gas passage through the heating chamber 24 from the inlet to the outlet. Therefore, when received, carrier gas flow from inlet 26 and will pass over the surface of the sample volume, which may assist in evaporation of the liquid sample as well as the transport of the evaporated sample to the heating chamber 24 and then to the oxidation chamber 26.

Heating or combustion chambers in previously designed analytical devices are typically configured to receive solid samples or to receive liquid samples in “boats” or other containers to prevent spillage. Therefore, the previously designed systems have not been optimized to automatically and/or continuously receive liquid samples. Embodiments of the present disclosure do not include sample “boats”. Instead, samples are received dir3ctly in the heating chamber 24 from the sample injection system 22.

After exiting the heating chamber 24, the vaporized sample travels to the oxidation chamber 28. In the oxidation chamber 28, compounds are removed from the gas stream that could either degrade the amalgamation trap or could cause re-oxidation of mercury. In one embodiment of the present disclosure, the oxidation chamber 28 is a catalytic oxidation chamber. As non-limiting examples, the catalytic oxidation chamber may include a Mn3O4/CaO-based catalyst, or other catalysts, such as catalysts based on Na2SO3 and CaCO3, CaSO4, or BaCO3. The catalyst helps to lower the heat requirement in the oxidation chamber 28 required to make sure combustion products from sample decomposition are fully oxidized. In addition, halogen, nitrogen, and sulfur oxide species can removed from the gas stream by the catalyst.

In another embodiment of the present disclosure, the oxidation chamber 28 does not include a catalyst, and only uses heating to oxidize other compounds. Without a catalyst, the temperature requirement in the oxidation chamber 28 is typically higher.

In general, maintaining a high temperature in the catalytic chamber (for example, to about 750 to 850 degrees C., preferably at least about 800° C.), will limit the possibility that mercury species could re-oxidize upon cooling. The oxidation chamber 28 may be an isothermal chamber, operating only at one temperature.

As the liquid sample evaporates in the heating chamber 24 and oxidizes in the oxidation chamber 28, the mercury is trapped on the mercury amalgamator 30. In embodiments of the present disclosure, the mercury amalgamation trap 30 is packed with gold-coated quartz sand or gold-coated glass beads. However, it should be appreciated that other traps may be within the scope of the present disclosure. To ensure accuracy in sample detection, trace mercury can be scrubbed from any gas entering the system 20, for example, either from carrier gas sources 26 or 42 by pulling the gas through similar mercury amalgamation trap 32 and 44.

Before the amalgamation trap 30 and after the oxidation chamber 28, the system 20 may include an optional dryer 60 to reduce water vapor entering the amalgamation trap 30. The advantage of using a dryer to reduce water vapor in the system 20 is to prevent water vapor exposure in the mercury amalgamator 30. In that regard, water vapor in a mercury amalgamator 30, such as a gold trap, may decrease the effectiveness of the trap. For example, water vapor may leach gold off the surface of the trap. In accordance with embodiments of the present disclosure, suitable dryers 60 include a membrane dryer, a coalescing filter, and/or a condenser.

The detector 40 will now be described in greater detail. In one embodiment of the present disclosure, the detector 40 may be a cold vapor atomic fluorescence spectrometer (CVAFS). In that regard, the inventors found that the atomic fluorescence (AF) technique provides better results for analyzing natural water samples than the atomic absorption (AA) technique. Specifically, atomic fluorescence (AF) is capable of more sensitive measurements and has a wider dynamic range that atomic absorption (AA), resulting in a lower detection limit. Atomic fluorescence (AF) detectors are currently required by the EPA methods for low level mercury, Methods 1631 and 245.7 (EPA 2002; EPA 2005), but have not been used for analysis by thermal decomposition in the past because of combustion-related interferences with the highly sensitive detector.

Previously developed systems using the atomic absorption (AA) technique, while effective for the analysis of solid samples such as fish tissue and other high mercury concentration solids, are limited in their usefulness for liquid analysis by the relatively poor sensitivity of atomic absorption spectrometry. Detection limits of previously developed systems range from 0.0015 ng to 0.005 ng. Because these systems accept relatively small samples (about 1 mL), the effective detection limit (about 1.5 to about 5 ng/L) is not low enough to quantify the majority of unpolluted natural waters.

Therefore, the system 20 described herein may include a combination of thermal decomposition in the heating chamber 24 and atomic fluorescence (AF) detection in the detector 40 (TD-AF). It should be appreciated, however, that embodiments of the present disclosure may also use atomic absorption (AA) detection, but these embodiments will have reduced detection sensitivity than a system using atomic fluorescence (AF) detection.

This combination for mercury analysis has been validated in recent years for petroleum products and minerals, using a water-based scrubber and a soda lime trap to remove interfering compounds, prior to pre-concentration on a gold trap and detection. However, the sensitivity of this TD-AF other system is limited, and requires frequent changing of water and soda lime traps to be feasible in a field deployed system. The system 20 described herein overcomes combustion-related interferences using a heated catalytic chamber 28, and can maintain high sensitivity in the detector 40 by using ultra-pure argon as a carrier gas.

In one embodiment of the present disclosure, the detector 30 is based on the Brooks Rand Model III CVAFS, but may include advancements that allow it to be operated in the field. The Model III and other CVAFS detectors currently in use are sensitive to large changes in temperature, making it infeasible to use them outdoors. The detector to be developed as part of this system is reengineered using less temperature-sensitive electronics, and also to be thermally insulated and contain a heating element, in order to maintain a relatively constant temperature and reduce temperature stabilizing time. It also includes more robust noise filtering electronics than are currently in use, allowing it to operate from a range of power sources, including batteries, generators, or standard alternating current. In addition, the detector includes data processing hardware capable of integrating peaks, storing data, and transferring results either to a locally-connected device or to a data transmitter. This hardware allows for data to either be manually downloaded periodically by the user or automatically transmitted via the cellular or satellite data networks.

Operation of the system 20 will now be described. First, a sample is received in the sample injection system 22 and pumped using pump 36 that pulls a carrier gas from carrier gas inlet 26 to deliver the sample to the heating chamber 24. As seen in FIG. 1, the carrier gas from carrier gas inlet 26 is run through a mercury trap 32 to remove any mercury from the carrier gas. As described above, the carrier gas may be air or any other inert or noble gas.

When the sample is received in the heating chamber 24, pump 36 is activated and valve 34 is open so as to allow for gas passage from the heating chamber 24 to the oxidation chamber 28 and the mercury amalgamator 30. Because pump 36 pulls gas through the system, the gas passage is in a one way direction.

When in the heating chamber 24, the sample is heated according to a heating sequence, a first temperature for receiving the sample, a second temperature for evaporating the sample, and a third temperature for decomposing any non-volatile mercury remaining in the heating chamber 24.

The system 20 may be run in either a two-step or a one-step operation schemes. A two-step heating process to separately detect volatile mercury and non-volatile mercury species will first be described. In accordance with the two-step heating process, the heating chamber 24 is heating to the evaporation temperature until the entire sample evaporates. At that time, valve 34 closes, separating the amalgamation trap 30 from the heating and oxidation chambers 24 and 28 upstream. The trap 30 is then heated, for example, under noble gas flow (such as ultra-pure argon gas) from a carrier gas inlet 42, desorbing all bound mercury into the detector 40. The gas flow may also pass through another trap 44 upon entering the system 20, to remove any mercury traces that may be present. Because the sample was only heated to the evaporation temperature, the mercury measured will only be dissolved gaseous mercury (Hg(0)), and not other forms of non-volatile mercury.

After the amalgamation trap 30 has been desorbed to the detector 40, the noble gas flow from the carrier gas inlet 42 shuts off and valve 34 reopens, reconnecting the amalgamation trap 30 to the sample heating and oxidation chambers 24 and 28.

The air pump 36 is reactivated, again pulling Hg-free gas (such as air) through the system 20. The heating chamber 24 will be rapidly raised to a temperature is the range of about 750 to 850° C., preferably at least about 800° C., to thermally decompose all Hg(II) species and reduce Hg(II) to Hg(0). The gas stream will be pulled through the oxidation chamber 28, which will be maintained at a constant temperature in the range of about 750 to 850° C., preferably 800° C., allowing for complete oxidation of combustion products and removal of reactive species such as halogens and nitrogen and sulfur oxides.

Mercury from this step is then collected on the amalgamation trap 30, which, again, is separated from the upstream chambers by the valve 34, and thermally desorbed into the detector 40 under noble gas flow. The mercury measured during this step represents non-volatile mercury species (Hg(II)). While the amalgamation trap 30 is being desorbed the second time, the sample heating chamber 24 will cool to about 150° C. and the pump will rinse the sample loop 22, readying the system to collect the next sample.

In accordance with a one-step heating process, all three heating steps are performed consecutively, and all mercury (including both volatile and non-volatile species) in the sample is trapped on the mercury amalgamator 30 and detected in a single detection step.

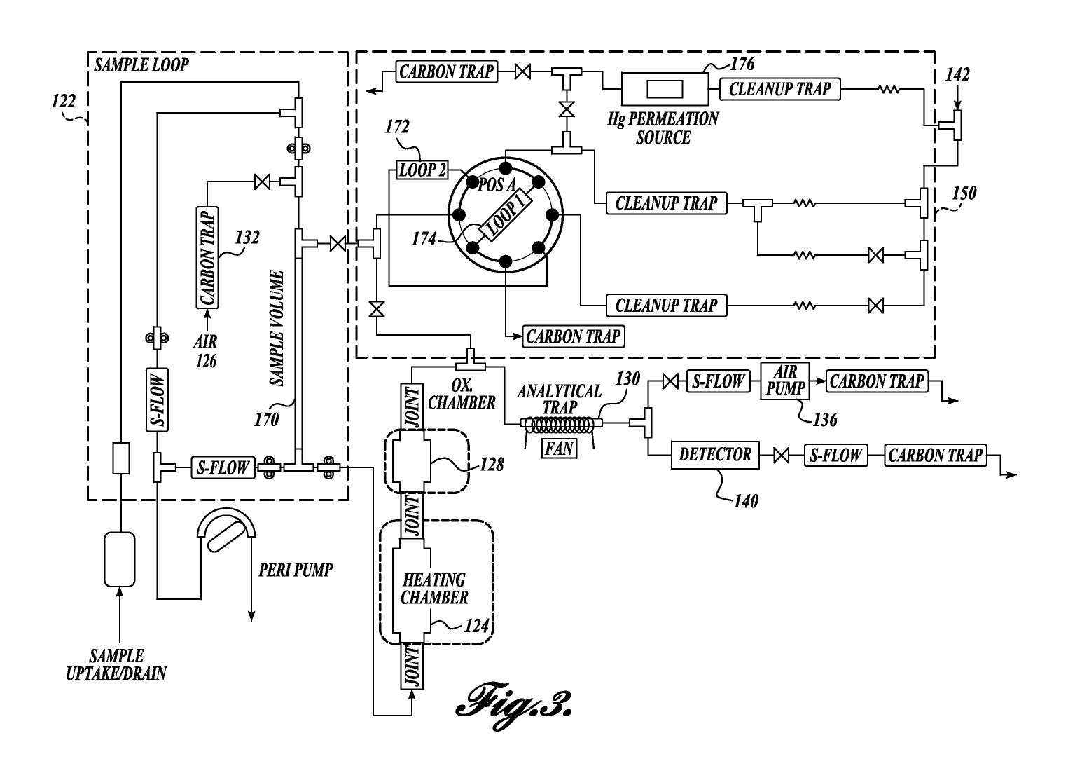

Referring now to FIGS. 2 and 3, a system 120 in accordance with another embodiment is provided. The embodiment of FIGS. 2 and 3 is substantially similar to the embodiment of FIG. 1, except for differences regarding the sample injection system and a calibration system. Like numerals are used to identify parts in FIGS. 2 and 3 as used in FIG. 1, except in the 100 series.

The system 120 of FIGS. 2 and 3 includes an exemplary sample loop injection system 122. The sample loop injection system 122 may be automated and is designed to collect water samples with minimal mercury carryover contamination between sampling. In the illustrated embodiment, the sample loop receives a sample in a fixed volume sample container 170, as opposed to a sample based on a fixed time period of sampling. The advantages of a fixed volume include the following. First, there is no need to control the flow rate or know the flow rate into the sample system, which is particularly advantageous in field sampling. Second, problems with inlet tubing are more prone to happen in field sampling. Therefore, if the inlet is clogged, the sample volume 170 will not fill, indicating an operational error.

Still referring to FIG. 2, a detailed schematic of an auto-calibration system 150 in accordance with one embodiment of the present disclosure is provided. The auto-calibration system 150 is designed to check accurate calibration of the system 120 over the course of a long, unattended deployment.

In one embodiment of the present disclosure, the auto-calibration system 150 may include one or more sample loops 172 and 174 of known volume in equilibrium with a chamber containing liquid mercury 176. The chamber 176 and sample loop 172 or 174 are held at a constant temperature, resulting in a fixed mass of mercury vapor in the loop.

Multi-port switching valves can be used to flush the loop with argon gas from the carrier gas inlet 142, then load the calibration mercury vapor onto the analytical trap 130. The trap 130 will then be desorbed, under Hg-free argon flow, into the detector 140, allowing the mercury vapor to be measured. This process will result in a calibration point. For additional calibration points, the mercury vapor loop volume can be diluted and injected onto the analytical trap 130 multiple times in sequence, before desorption. In this way, the system 120 will be calibrated across the linear range of the detector 140, at user-determined intervals.

The systems and methods described herein have many advantages over previously developed systems. The system will save users significant expense and effort by eliminating the requirement to transport samples back to the laboratory for analysis. It will also eliminate significant contamination risk by removing the need for sample containers. In addition, in the same way that automated laboratory mercury analyzers reduce contamination by eliminating the need for personnel to introduce samples into the analytical system, the system will reduce contamination risk by removing personnel from the collection of field samples.

The system is useful for monitoring surface water and groundwater at ambient and contaminated mercury levels. For example, it may benefit the public in at least the following ways discussed below.

1. It provides a cost-effective, long term monitoring solution for the characterization and remediation of groundwater mercury plumes.

2. It generates real-time data for surface and groundwater systems that can be made publicly accessible via the Internet.

3. It lowers the cost of environmental monitoring.

4. By collecting regular, high-frequency measurements, the system exposes biogeochemical processes not currently visible due to the low temporal resolution of most sampling campaigns.

By providing a cost-effective means of characterizing and monitoring groundwater contamination, the system described herein may facilitate more targeted clean-up efforts in areas of subsurface mercury contamination. Remediating subsurface mercury contamination has human and ecosystem health benefits for those living downstream of the contaminated system, and reducing the cost of monitoring has benefits for the agency responsible for the remediation.

Through its capacity to produce real-time data, the system can be used as a tool to increase public understanding of environmental contamination and increase awareness of the health of local ecosystems. Automated mercury monitors could be deployed in a network similar to the U.S. Geological Survey's stream gauge network, for example, and could also provide real-time data via the Internet. Such an infrastructure would make it easy for the public to understand how mercury contamination affects local ecosystems.

Because the field-deployable mercury monitor will significantly reduce the costs associated with field sampling and analysis, it will allow more cost-effective environmental regulatory compliance monitoring, and will likely enable such monitoring to be carried out in more places. A lower operating cost also helps ensure that monitoring programs will be able to exist for the long term by lowering the risk that they will be discontinued due to budget constraints.

By collecting high frequency measurements of mercury concentrations in water, the mercury monitor system will make it possible to observe environmental trends with high temporal resolution. This level of study is particularly important in many rivers, where it has been demonstrated that mercury concentrations spike during high flow events (the first flush principle), sometimes accounting for the majority of a system's annual mercury load. In a recent report, storm driven fluxes were identified as a dominant contributor to the annual discharge of mercury from a specific site, but the lack of high frequency measurements makes identifying factors controlling these fluxes difficult.

While high frequency data exist for some systems, the cost of acquiring such data is prohibitive for most investigators. By making it feasible to monitor such rapid trends more often and in more systems, the proposed monitor will greatly enhance our understanding of the mercury cycle.

The systems and methods described herein may be portable, field-deployed, or site-deployed mercury monitoring systems and methods. However, it should be appreciated that lab mercury monitoring systems and methods are also within the scope of the present disclosure. As seen in FIG. 4, a schematic of a control system for the mercury monitoring system is provided.

Therefore, the system described herein is a computer-controlled mercury analysis system that automatically collects water samples from an environmental water body and analyze them for Hg(0) and Hg(II) via thermal decomposition and cold vapor atomic fluorescence spectrometry. In one embodiment, the system has a lower detection limit of about 0.5 ng/L and a sample throughput of up to about 12 samples/hour. However, it should be appreciated that other detection limits and maximum sample throughputs are within the scope of the present disclosure

While illustrative embodiments have been illustrated and described, it will be appreciated that various changes can be made therein without departing from the spirit and scope of the disclosure.

1. A mercury monitoring system for detecting an amount of total mercury in a liquid sample, the system comprising:
(a) a sample inlet for receiving a liquid sample;
(b) a heating chamber in direct fluid communication with the sample inlet, wherein the heating chamber is configured for evaporating the entire liquid sample for detection in a single heating cycle, the heating chamber including a fluid reservoir to receive and contain the entire liquid sample prior to evaporation;
(c) an oxidation chamber for oxidizing the evaporated sample;
(d) a mercury amalgamator for trapping elemental mercury; and
(e) a mercury detector.

2. The system of claim 1, further comprising a flow of gas delivered to the heating chamber from a gas source.

3. The system of claim 1, wherein the sample inlet is a sample injection system.

4. The system of claim 1, wherein the sample inlet is configured to receive a fixed volume sample.

5. The system of claim 4, wherein the fixed volume sample has a volume selected from the group consisting of in the range of about 1.5 mL to about 10 mL and in the range of about 1.5 mL to about 20 mL.

6. The system of claim 1, wherein the fluid reservoir includes a plumbing trap for receiving the liquid sample.

7. The system of claim 6, wherein the plumbing trap includes an inlet line at a first elevation, the fluid reservoir at a second elevation lower than the first elevation, and an outlet line at a third elevation higher than the second elevation.

8. The system of claim 6, wherein the heating chamber is configured to contain the entire liquid sample prior to evaporating the liquid sample.

9. The system of claim 1, wherein the heating chamber does not receive a sample contained in a boat.

10. The system of claim 1, wherein the heating cycle includes a first sample receipt temperature and a second sample evaporation temperature.

11. The system of claim 1, wherein the heating cycle further includes a third sample decomposition temperature.

12. The system of claim 1, wherein the system does not use reagents for sample decomposition.

13. The system of claim 1, further comprising an internal calibration system.

14. The system of claim 1, wherein the mercury detector is a CVAFS detector.

15. The system of claim 1, wherein the mercury amalgamator is a gold amalgamation trap.

16. The system of claim 1, further comprising a dryer for water vapor removal.

17. A mercury monitoring system for detecting an amount of total mercury in a liquid sample, the system comprising:
(a) a sample inlet for receiving a liquid sample;
(b) a heating chamber in direct fluid communication with the sample inlet, wherein the heating chamber is configured for evaporating the entire liquid sample for detection in a single heating cycle, the heating chamber including a includes a plumbing trap for receiving the liquid sample, wherein the plumbing trap includes an inlet line at a first elevation, a fluid reservoir at a second elevation lower than the first elevation, and an outlet line at a third elevation higher than the second elevation;
(c) an oxidation chamber for oxidizing the evaporated sample;
(d) a mercury amalgamator for trapping elemental mercury; and
(e) a mercury detector.

18. A method of detecting an amount of total mercury in a liquid sample, the method comprising:
(a) collecting a liquid sample in an inlet line;
(b) transferring the entire liquid sample from the inlet line directly to the sample decomposition chamber;
(c) containing the liquid sample in the sample decomposition chamber, to allow a flow of gas to flow from the gas source into the heating chamber when the liquid sample is contained;
(d) heating the liquid sample to evaporate the liquid;
(e) transferring the evaporated sample through a catalytic oxidation chamber to remove combustion products; and
(f) trapping the volatilized mercury.

19. The method of claim 18, further comprising releasing the mercury from the trap and detecting the mercury.

20. The method of claim 18, further comprising decomposing any non-volatile components in the sample.
